(19) United States
(12) Patent Application Publication
(10) Pub. No.: US 2011/0049391 A1
(43) Pub. Date: Mar. 3, 2011

(54) Storage Container Having Sterilizing Function by Using Ultraviolet Rays

(76) Inventor: Cheng-Pu Yang, Sihu Township (TW)

(21) Appl. No.: 12/551,698

(22) Filed: Sep. 1, 2009

(51) Int. Cl. A61L 2/10

(52) U.S. Cl. 250/492.1

(57) ABSTRACT

A storage container includes a container body having an inner portion provided with a receiving chamber, a top cover removably mounted on the container body, a circuit board mounted in the top cover, an ultraviolet light mounted on the circuit board and directed toward the receiving chamber of the container body, a control switch mounted on the circuit board and electrically connected with the ultraviolet light, at least one battery mounted on the circuit board and electrically connected with the control switch and the ultraviolet light, and a push button mounted on the top cover and contacting with the control switch. Thus, the ultraviolet light provides a sterilizing function to the articles placed in the receiving chamber of the container body so as to achieve a sanitary purpose.

### BACKGROUND OF THE INVENTION

1. Field of the Invention

The present invention relates to a container and, more particularly, to a storage container to receive and store articles, such as forks, spoons, chopsticks and the like.

2. Description of the Related Art

A conventional storage container comprises a container body having an inner portion provided with a receiving chamber and a top cover removably mounted on the container body. In use, when articles, such as forks, spoons, chopsticks and the like, are placed in the receiving chamber of the container body, the top cover is mounted on the container body to close the receiving chamber of the container body and to cover the articles. However, the receiving chamber of the container body is sealed by the top cover so that the articles are disposed at a wet and closed condition, so that bacteria are deposited on the articles, thereby easily incurring a sanitary problem.

### BRIEF SUMMARY OF THE INVENTION

In accordance with the present invention, there is provided a storage container, comprising a container body having an inner portion provided with a receiving chamber, a top cover removably mounted on the container body to close the receiving chamber of the container body, a circuit board mounted in the top cover, an ultraviolet light mounted on the circuit board and directed toward the receiving chamber of the container body, a control switch mounted on the circuit board and electrically connected with the ultraviolet light to turn on or off the ultraviolet light, at least one battery mounted on the circuit board and electrically connected with the control switch and the ultraviolet light, and a push button mounted on the top cover and contacting with the control switch to control operation of the control switch.

The primary objective of the present invention is to provide a storage container having a sterilizing function by using ultraviolet rays.

According to another objective of the present invention, the ultraviolet light provides a sterilizing function to the articles placed in the receiving chamber of the container body so as to achieve a sanitary purpose.

According to a further objective of the present invention, the volume of the circuit board is reduced to the minimum by provision of the mercury battery and the ultraviolet LED light so that the storage container has a smaller volume and lighter weight to facilitate packaging, storage and transportation of the storage container.

Further benefits and advantages of the present invention will become apparent after a careful reading of the detailed description with appropriate reference to the accompanying drawings.

### BRIEF DESCRIPTION OF THE SEVERAL VIEWS OF THE DRAWING(S)

FIG. 1 is a perspective view of a storage container in accordance with the preferred embodiment of the present invention.

FIG. 2 is an exploded perspective view of the storage container as shown in FIG. 1.

FIG. 3 is a front cross-sectional view of the storage container as shown in FIG. 1.

FIG. 4 is a schematic operational view of the storage container as shown in FIG. 3 in use.

### DETAILED DESCRIPTION OF THE INVENTION

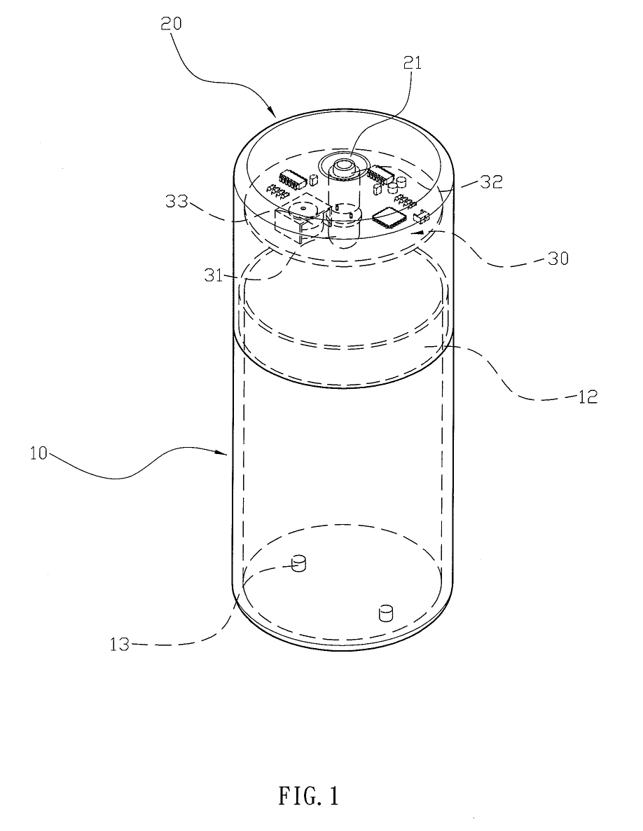

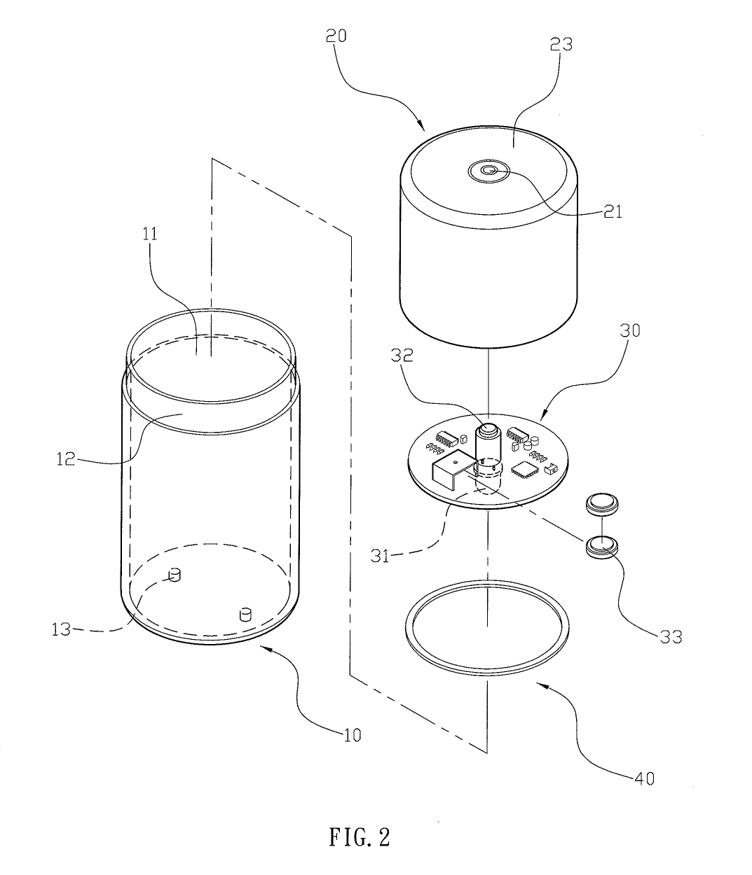

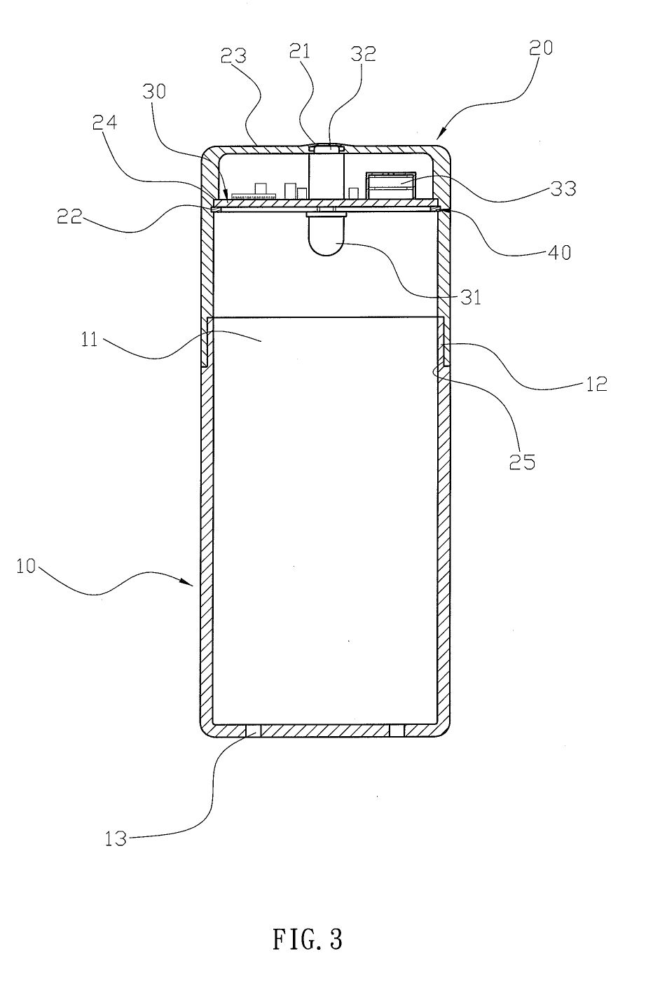

Referring to the drawings and initially to FIGS. 1-3, a storage container in accordance with the preferred embodiment of the present invention comprises a container body 10 having an inner portion provided with a receiving chamber 11, a top cover 20 removably mounted on the container body 10 to close the receiving chamber 11 of the container body 10, a circuit board 30 mounted in the top cover 20, an ultraviolet light 31 mounted on the circuit board 30 and directed toward the receiving chamber 11 of the container body 10, a control switch 32 mounted on the circuit board 30 and electrically connected with the ultraviolet light 31 to turn on or off the ultraviolet light 31, at least one battery 33 mounted on the circuit board 30 and electrically connected with the control switch 32 and the ultraviolet light 31, and a push button 21 mounted on the top cover 20 and contacting with the control switch 32 to control operation of the control switch 32.

The container body 10 has a U-shaped cross-sectional profile. The container body 10 has an open upper end provided with a stepped mounting portion 12 and has a closed bottom portion provided with at least one draining hole 13 connected to the receiving chamber 11.

The circuit board 30 is located at a mediate portion of the top cover 20. The circuit board 30 is fixed in the top cover 20 by a retaining member 40. The circuit board 30 is located above the retaining member 40. The top cover 20 has an inner wall provided with a retaining groove 22 to retain the retaining member 40. The retaining member 40 is made of a waterproof rubber and has a ring shape. The retaining groove 22 of the top cover 20 has an annular shape. The inner wall of the top cover 20 is further provided with a stepped fixing recess 24 to receive the circuit board 30. The fixing recess 24 of the top cover 20 is located above and connected to the retaining groove 22.

The top cover 20 has an inverted U-shaped cross-sectional profile. The top cover 20 has a closed top portion 23 abutting an upper end of the control switch 32 and an open lower end provided with a stepped mounting hole 25 mounted on the mounting portion 12 of the container body. The push button 21 is made of soft pad and is mounted on the top portion 23 of the top cover 20. Preferably, the push button 21 is located at a central portion of the top portion 23 of the top cover 20.

The control switch 32 has an upper end abutting the top portion 23 of the top cover 20 and the push button 21 and has a lower end mounted on a top of the circuit board 30. The control switch 32 is electrically connected with the circuit board 30.

The ultraviolet light 31 is a light emitting diode (LED) and protrudes downward from a bottom of the circuit board 30. The ultraviolet light 31 is electrically connected with the circuit board 30. The ultraviolet light 31 and the control switch 32 are located at two opposite sides of the circuit board 30. The battery 33 is preferably a mercury battery.

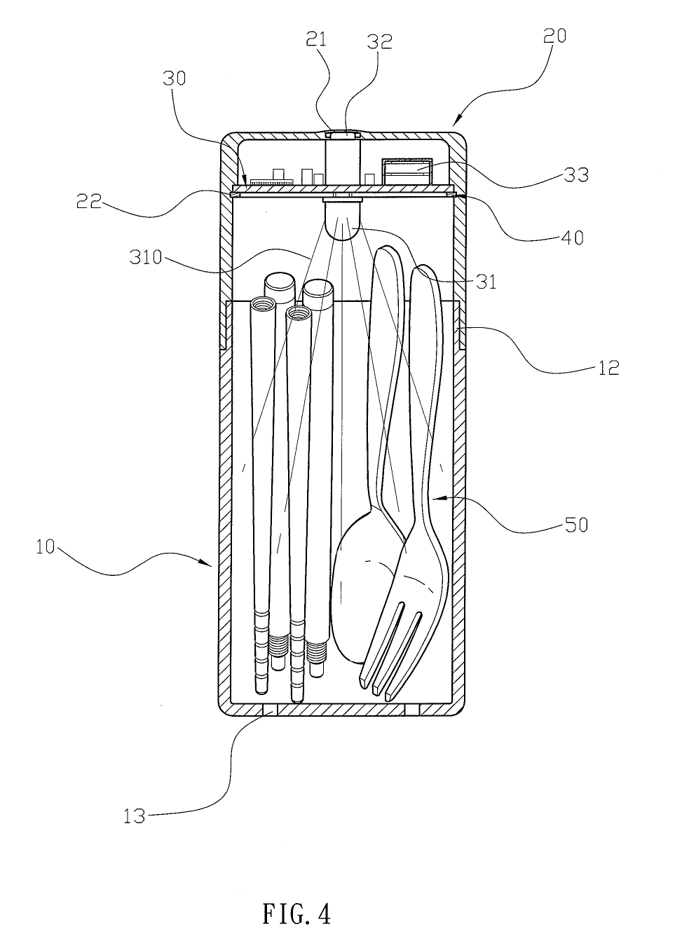

In operation, referring to FIG. 4 with reference to FIGS. 1-3, when articles 50, such as forks, spoons, chopsticks and the like, are placed in the receiving chamber 11 of the container body 10, the top cover 20 is mounted on the container body 10 to close the receiving chamber 11 of the container body 10 and to cover the articles 50. Then, the push button 21 is pressed by a user to control operation of the control switch 32 so as to turn on or off the ultraviolet light 31. In such a manner, after the ultraviolet light 31 is turned on, the ultraviolet light 31 emits ultraviolet rays 310 toward the receiving chamber 11 of the container body 10 so as to provide a sterilizing function to the articles 50 placed in the receiving chamber 11 of the container body 10. At this time, the water on the articles 50 can flow through the draining hole 13 of the container body 10 and is drained outwardly from the draining hole 13 of the container body 10.

In another preferred embodiment of the present invention, the storage container further comprises a timer mounted on and electrically connected with the circuit board 30 to control the operation time of the ultraviolet light 31.

Accordingly, the ultraviolet light 31 provides a sterilizing function to the articles 50 placed in the receiving chamber 11 of the container body 10 so as to achieve a sanitary purpose. In addition, the volume of the circuit board 30 is reduced to the minimum by provision of the mercury battery 33 and the ultraviolet LED light 31 so that the storage container has a smaller volume and lighter weight to facilitate packaging, storage and transportation of the storage container.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

1. A storage container, comprising:
a container body having an inner portion provided with a receiving chamber;
a top cover removably mounted on the container body to close the receiving chamber of the container body;
a circuit board mounted in the top cover;
an ultraviolet light mounted on the circuit board and directed toward the receiving chamber of the container body;
a control switch mounted on the circuit board and electrically connected with the ultraviolet light to turn on or off the ultraviolet light;
at least one battery mounted on the circuit board and electrically connected with the control switch and the ultraviolet light;
a push button mounted on the top cover and contacting with the control switch to control operation of the control switch.

2. The storage container of claim 1, wherein the container body has a closed bottom portion provided with at least one draining hole connected to the receiving chamber.

3. The storage container of claim 1, wherein the circuit board is fixed in the top cover by a retaining member.

4. The storage container of claim 3, wherein the top cover has an inner wall provided with a retaining groove to retain the retaining member.

5. The storage container of claim 1, wherein
the container body has a U-shaped cross-sectional profile;
the top cover has an inverted U-shaped cross-sectional profile.

6. The storage container of claim 1, wherein
the container body has an open upper end provided with a stepped mounting portion;
the top cover has an open lower end provided with a stepped mounting hole mounted on the mounting portion of the container body.

7. The storage container of claim 1, wherein the ultraviolet light and the control switch are located at two opposite sides of the circuit board.

8. The storage container of claim 1, wherein the circuit board is located at a mediate portion of the top cover.

9. The storage container of claim 3, wherein the retaining member is made of a waterproof rubber.

10. The storage container of claim 4, wherein the retaining member has a ring shape.

11. The storage container of claim 10, wherein the retaining groove of the top cover has an annular shape.

12. The storage container of claim 4, wherein the inner wall of the top cover is further provided with a stepped fixing recess to receive the circuit board.

13. The storage container of claim 12, wherein
the circuit board is located above the retaining member;
the fixing recess of the top cover is located above and connected to the retaining groove.

14. The storage container of claim 1, wherein the ultraviolet light is a light emitting diode (LED).

15. The storage container of claim 1, wherein the top cover has a closed top portion abutting an upper end of the control switch.

16. The storage container of claim 1, wherein the push button is made of soft pad.

17. The storage container of claim 1, wherein
the control switch is electrically connected with the circuit board;
the ultraviolet light is electrically connected with the circuit board.

18. The storage container of claim 1, wherein the battery is a mercury battery.

19. The storage container of claim 15, wherein
the push button is mounted on the top portion of the top cover;
the push button is located at a central portion of the top portion of the top cover;
the control switch has an upper end abutting the top portion of the top cover and the push button and has a lower end mounted on a top of the circuit board.

20. The storage container of claim 1, wherein the ultraviolet light protrudes downward from a bottom of the circuit board.
